(19) United States
(12) Patent Application Publication
(10) Pub. No.: US 2017/0281701 A1
(43) Pub. Date: Oct. 5, 2017

(54) Formulation for the Nebulization of Oil Based Substances Suitable for Use with a Vibrating Mesh Nebulizer

(71) Applicant: Catherine Ket Wah Kan, Toronto (CA)

(72) Inventor: Catherine Ket Wah Kan, Toronto (CA)

(21) Appl. No.: 15/479,251

(22) Filed: Apr. 4, 2017

Related U.S. Application Data
(60) Provisional application No. 62/318,213, filed on Apr. 5, 2016.

(51) Int. Cl. A61K 36/185; A61M 11/00; A61K 9/127; A61K 9/00; A61K 31/352; A61K 31/05

(52) U.S. Cl. CPC A61K 36/185; A61K 31/352; A61K 31/05; A61K 9/127; A61K 9/0078; A61M 11/005

(57) ABSTRACT

Several variations of formulations are used as a water base solution to disperse oil soluble pharmaceuticals, including cannabis extract, suitable for use with a VMN. Common elements include added small amount of sodium electrolytes to facilitate the nebulization process and ethanol in controlled amount as needed to dilute or dissolve thick waxy substances such as found in some cannabis extracts. Surfactants, co-surfactants and/or emulsifiers to include but not limited to Acconon™, ethanol, hydroxylated soy lecithin and/or sodium lauryl sulfate are used. The ingredients are sonicated to obtain a uniform respirable liposomal suspension that could be delivered in low micron range using a VMN for efficient absorption into the blood stream by oral inhalation.

### BACKGROUND OF THE INVENTION

### Field of the Invention

The present invention generally relates to the base solution used as a carrier for oil based substances such as but not limited to cannabis extracts (Appendix A) and, more specifically, to formulations for the delivery of such extracts in an ultrafine liposomal water base mist for oral inhalation using a device such as but not limited to a portable hand held vibrating mesh nebulizer (VMN).

### 2. Description of the Prior Art

A liposomal water based formulation has been proposed with added electrolytes to specifically improve the nebulization process when used with a hand held vibrating mesh nebulizer (VMN). Such a formulation could be used with but not limited to cannabis extracts. Such a formulation could be used with but not limited to VMN. When combined together, this formulation is required for the nebulization of cannabis extracts using a VMN.

Known liposomal formulations have been used to deliver oil soluble pharmaceuticals in the past. For example, liposomal aerosol formulations have been disclosed in U.S. Pat. Nos. 7,341,739; 6,346,233 and 6,090,407 to deliver various anti-cancer drugs by oral inhalation. VMN have also been available for many years but have only been used for aqueous solutions that can pass through the 2,000 to 3,000 nano holes within a generally 6 mm sized diameter of a mesh made of precious metals as used with this invention. Cannabis extracts has thus far never been formulated to he delivered through a VMN.

The scientific knowledge of the cannabis plant and its application is going to significantly proliferate in this decade. Already we are today capable of producing and isolating the exact molecule or combinations of molecular compounds to specifically target an ailment using cannabinoids identified within the cannabis plant.

Until recent years, the administration of cannabis has mostly been achieved by smoking the cannabis plant. Today, various extraction methods have been developed in order to isolate the desired components within the cannabis plant in targeting a specific therapeutic response. Also there is evidence, called the entourage effect, that the whole plant extract confers benefits beyond the sum of its parts. The extraction process has been refined to such a degree that it reaches purity of up to 90%. This would mean less waxy matrix is left even with whole plant extracts. Such extracts are often used as is in “vap” pens to be described below or compounded into different dosage forms to be swallowed, instilled, applied, inserted or inhaled. It is this inhaled route of administration that is the focus of this invention.

Recent methods have sought to find new ways to deliver cannabinoids to a patient by inhalation that would mitigate the adverse effect of inhaling unhealthy tars and associated carcinogens into their lungs when they smoke the cannabis plant. Four alternatives exist and our proposed invention would be the fifth. CannaNeb, a company out of San Diego, Calif. has patented a jet nebulizer that nebulizes the thick cannabis extract directly without further dissolution and/or dilution. Syqe Medical out of Israel has developed a metered dose inhaler that delivers vaporized cannabis granules. There are many brands of metered dose inhalers such as Mystabis, PUFFit or Vapen Clear that uses a pressurized canister with a propellant such as hydrofluroalkane to expel a calibrated amount of cannabis extract in an aerosol. The fourth delivery method which has become quite popular is “vaping” a process of inhaling the vapor produced from heating but not burning the cannabis bud or heating cannabis extract to about 200° C.+ to produce vapor using propylene glycol and/or glycerol as a solvent vehicle.

Other dosage forms on the market include administering the cannabinoids to the sublingual or buccal mucosa, via enemas or solid dosage forms such as gels, capsules, tablets, pastilles, lozenges, infused food substance as well as by topical application. All of the aforementioned methods of cannabis delivery have inherent shortcomings based on the pharmacokinetics of drug absorption or thermal alteration when heat is applied.

### SUMMARY OF THE INVENTION

The objective of the present invention is to provide a respirable formulation that can carry the whole cannabis plant extract or components thereof to be delivered in a precise dosing format for oral inhalation, either in a unit dose or a controlled fitratable multi-dose form at suitable for use with a VMN.

The present invention is proposed to solve the following problems:

1: Enable the delivery of a lower dose of the cannabis extract for the desired effect compared to smoking or vaping due to the inherent efficiency of the VMN (particle size <5 microns, Mass Median Aerodynamic Diameter (MMAD) of 2.1, no waste and, cascade impactor result ≈80% in the respirable fraction of between 1-5 μm FIG. 1,2, 4,5).

2: No heating is required and no thermal energy is generated using the VMN, preserving the integrity of the active substances. What you put in is what you get.

3: Patient response is within 5 minutes which facilitates quick and precise symptom to dose titration.

4: Each oral inhalational dose generates no smoke and no exhale.

5: The dose is delivered without smell, without noise and without complicated equipment such as respiratory tubing, spacer or a compressor.

6: Ease of administration without a high degree of hand breath coordination.

Four variable pharmaceutical formulations suitable to nebulize cannabis extract using VMN is provided herein for pulmonary delivery. The formulations comprise of a water-based liquid, which includes a mixture of a cannabis oil extract to be added, having a known amount of a medicinal cannabis compound emulsified in an aerosol precursor. When sonicated, the aerosol precursor forms a homogenous liposomal micro-emulsion with the cannabis extract using surfactants, co-surfactants and/or emulsifiers such as but not limited to Acconon™, ethanol, hydroylated soy lecithin and/or sodium butyl sulfate (Table 1). Through such a process, a stable liposomal water based suspension is formed (FIG. 5). The emulsified solution can be nebulized into an ultraline inhalable mist for deep pulmonary deposition using a small hand held VMN. (FIG. 5).

These formulations enable the delivery of precise cannabis components in the desired strength for immediate absorption into the blood stream through the pulmonary route. This is achieved without the application of any heat source, without combustion, without smoking and by-passing the gut and the first pass metabolism of the liver, allowing approximately 80% of the active substrates available for pulmonary deposition (FIGS. 1, 2, 4. 5).

The medicinal cannabis compound may include cannabinoids, terpenes, flavonoids, phytosterols, and/or other medicinally relevant compounds found in cannabis (Appendix. A). The medicinal cannabis can also be synthetically derived or biotechnologically engineered, thermally or chemically altered such as but not limited to the process of decarboxylation of tetrahydrocannabinolic acid (THCA) to tetrahydrocannabinol (THC). Since the formulation contains a fat soluble substrate, hemp oil or cannabis extract, additional fat soluble substrates besides what is mentioned in Appendix A could also be added to the mix as a supplement or a diluent. Examples could be but not limited to lipophilic substrates such as lycopene, vitamin E or lutein where added benefits could be conferred as an antioxidant against free radicals or in the case of tocotrienol from Vitamin E, conferring neuronal protection, cholesterol reduction and protection against stroke-associated brain damage. (32, 33, 34).

The ability to deliver the formulation of cannabis extract with or without other supplemental lipophilic substrates in a ready to be absorbed state without further alteration by the end user or complicated equipment set up allows the precise tailoring of the active moieties of the cannabis containing compound for a targeted effect by the end user. The medicinal cannabis compound may be administered to or by a patient on an as needed titratable dosing basis or in a controlled unit dose format.

### BRIEF DESCRIPTION OF THE FIGURES

Other objects and advantages of the present invention will be readily apparent to those skilled in the art from the following description of the figures in which an exemplary embodiment of the invention is shown.

FIG. 1 is a diagram showing aerosol particle sizes and corresponding pulmonary deposition;

FIG. 2 is a diagram showing aerosol particle size and where they can reach in the lung;

FIG. 3 are diagrams illustrating traditional nebulizers;

FIG. 4 is a histogram illustrating particle size performance of a vibrating mesh nebulizer used

FIG. 5 is a picture of the VMN device used for this invention and an example of Formula #2 in making a 3% Cannabis Extract formulation

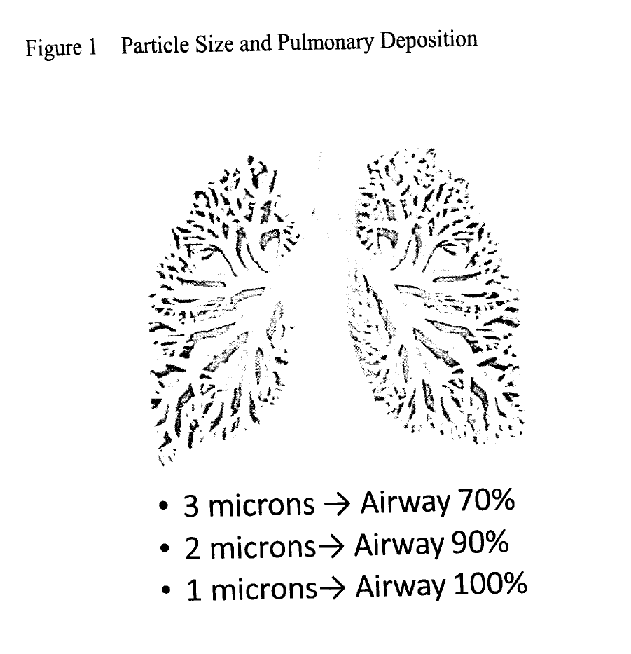

### DETAILED DESCRIPTION

Definitions

An aerosol precursor is a substance that is capable of being dispersed into a system of solid or liquid particles suspended in air or other gaseous environment. Aerosols can vary in size and composition depending on the substrate used and the delivery system deployed.

The “entourage effect” refers to the interaction of various compounds in cannabis. Cannabis, unlike most modern day medicine, contains a wide range of chemical compounds. Scientists have identified over 60 unique molecules in cannabis known as cannabinoids, which include THC and CBD. Many other non-cannabinoid compounds are produced by the plant that also have regulatory effects. For example, terpenes, the molecules responsible for marijuana's smell, have been shown to block some cannabinoid receptor sites in the brain while promoting cannabinoid binding in others. As a result, terpenes are believed to affect many aspects of how the brain takes in THC or cannabidiol (CBD), while offering various therapeutic benefits of their own. While THC, the psychoactive molecule within cannabis has gotten most of the attention, studies suggest many of the compounds in cannabis work together to produce a synergy of effects. This is known as the “entourage effect”.

Hemp oil or hempseed oil is an oil extracted from the hemp plant. All plants in the Cannabis genus can produce the oil, but usually only industrial hemp is used to make hemp oil. Industrial hemp is a hemp variety which has been cultivated specifically for industrial production, and it has a minimum of psychoactive substances associated with the genus, most notably A-9-tetrahydrocannabinol (THC). The oil could be used to dilute a concentrated cannabis extract.

Cannabis, also known as marijuana among several other names, is a preparation of the Cannabis plant intended for use as a psychoactive drug or medicine.

A single unit dose is herein defined as a maximum dose of the medication that can be taken at any one time or within a specified dosage period.

The term titratable dose is defined as meaning that the patient is provided with a medication that is in such a form that smaller doses of the formulation can be taken. Titration of doses is beneficial to the patient as they are able to take smaller doses of the medication until the drug is efficacious. Not all patients will require exactly the same dose of medication. Dependent factors could include but not limited to body size, metabolic rate, renal and/or hepatic clearance, genetics, and the state of the drug/tissue interface during absorption. The benefits of delivering cannabis extracts using VMN over other dosage forms are evident as smaller aliquots could be used and without the lengthy wait times of up to 2 hours for peak response as in the oral route.

Decarboxylation is a chemical reaction that releases carbon dioxide (CO₂). This refers to a chemical reaction that takes place in which carboxylic acids loose a carbon atom from a carbon chain. This process for example, converts THCA (acid form of THC found in abundance in growing and harvested cannabis and is a biosynthetic precursor of THC) to THC. THC is the active compound with many medicinal and psychoactive effects. When the cannabis plant dries, it very slowly begins to decarboxylate and converts THCA to THC.

MMAD refers to mass median aerodynamic diameter defined as the diameter at which 50% of the particles by mass are larger and 50% are smaller.

A cascade impactor is strictly a measurement-related device. In addition to measuring the range of substances moved through an opening by aerosol, the impactor can also be used to determine the particle size of the distributed substance.

Formulations

Sample formulations are set forth in Table 1 below used to deliver a continuous or an intermittent nebulization of cannabis extract using a palladium mesh VMN capable of generating mean particle sizes of under 5 microns. The cannabis or hemp oil extract in various concentrations is added to a water base solution containing surfactants, co-surfactants and/or emulsifiers and then put through a simple sonicator or ultrasonic homogenizer. A more sophisticated fluidizer could also be used to enhance the stability of the emulsion. A gentle agitation of the solution prior to use is recommended to prevent separation upon long term standing.

TABLE 1
Sample Formulations

Formulation #1
5% Acconon ™
5% Hemp Oil or Cannabis Extract
89.75% Water
0.25% Sodium Lauryl Sulfate
Clear solution with powdery liposomes
Formulation #2
5% Hydroxylated Soy
Lecithin 10% Ethanol
5% Hemp Oil or Cannabis Extract
0.25% Sodium Lauryl Sulfate
79.75% Water
Clear light milky or slightly yellowish milky liposomes
Formulation #3
Same as #2, except replace the water with 0.1% gellan gum in water
with 5 mM CaCl2 Gellan solution made with 200 ml water, 200 mg High
Acyl Gellan Gum, 111 mg CaCl2. Mix gellan with cold water, medium to
high shear mixing. Heat to 194 degree F., add CaCl2. Cool to RT and use.
Formulation #4
5% Hydroxylated Soy Lecithin
5% Ethanol
5% Hemp Oil or Cannabis Extract
0.9% Sodium Chloride
84.1% Water

### Sample Processing of Cannabis Extraction is illustrated in Table 2

TABLE 2

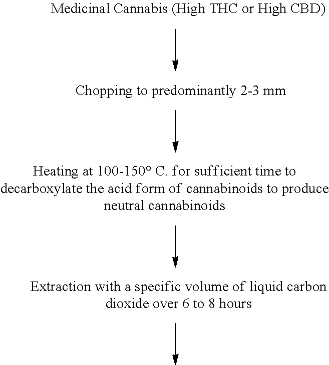

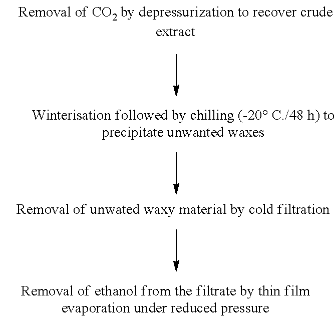

Various Modes of Cannabis Intake

Smoking Cannabis

Like nebulization, smoking cannabis results in a more rapid onset of action, higher blood levels of cannabinoids, and a shorter duration of pharmacodynamic effects compared to oral administration (1). The amount of Δ⁹-THC delivered from cannabis cigarettes is variable (1). Factors contributing to this variability include the plant source together with the efficiency and method of smoking used by the patient (1, 3). Smokers often claim that they can titrate their Δ⁹-THC intake by adapting their smoking behavior to obtain the desired effect (4, 5). Δ⁹-THC absorption by smoke inhalation is fast and variable, with a bioavailability of 2-56% depending on depth of inhalation, puff duration, and breath hold (6, 7). In practice, a maximum of 25-27% of the THC content in a cannabis cigarette is absorbed or delivered to the systemic circulation from the total available amount (2, 8).

Vaporized Cannabis

Vaporization of cannabis generally requires a heat source in the form of a heating coil or plate. For drug particles to be absorbed by the pulmonary route, they must be carried deep into the alveolar sacs and into the alveoli terminal of the bronchioles where there are over 100 meter square of absorptive surface area in an adult. Vaporization allows this to happen by heating the cannabis extract to release vapor and often using propylene glycol and/or glycerol as a solvent vehicle.

The potential advantages of vaporization include the formation of a smaller quantity of toxic by-products such as carbon monoxide, polycyclic aromatic hydrocarbons (PAHs), and tar, as well as a more efficient extraction of Δ⁹-THC from the cannabis material (4, 9, 10, 11, 12). The subjective effects and plasma concentrations of Δ⁹-THC obtained by vaporization of cannabis are comparable to those obtained by smoking cannabis, with absorption being somewhat faster with the vaporizer compared to smoking, according to one study (4). In addition, the study reported that the vaporizer was well tolerated with no reported adverse effects, and was preferred over smoking by the test subjects (4). While vaporization has been reported to be amenable to self-titration (as has been claimed for smoking) (4, 11), the proper use of the vaporizer for optimal administration of cannabis for therapeutic purposes needs to be established in more detail (12). The amount and type of cannabis placed in the vaporizer, the vaporizing temperature and duration of vaporization, and the balloon volume are some of the parameters that can affect the delivery of Δ⁹-THC (11).

Oral

Whereas the central nervous system and physiological effects occur within minutes by the smoking route or by vaporization (13, 14), these effects proceed much slower in the case of oral ingestion (14, 15). Oral administration results in a slower onset of action, lower peak blood levels of cannabinoids, and a longer duration of pharmacodynamic effects compared to smoking (1). The psychotropic effect or “high” occurs much more quickly by the smoking than by the oral route, which is the reason why smoking appears to be the preferred route of administration by many, especially recreational users (16).

For orally administered prescription cannabinoid medicines such as synthetic Δ⁹-THC (dronabinol, marketed as Marinol®), only 10-20% of the administered dose enters the systemic circulation indicating extensive first-pass metabolism (5)

Δ⁹-THC can also be absorbed orally by ingestion of foods containing cannabis (e.g. butters, oils, brownies, cookies), and teas prepared from leaves and flowering tops. Absorption from an oral dose of 20 mg Δ⁹-THC in a chocolate cookie was described as slow and unreliable (3), with a systemic availability of only 4-12% (3).

Oro-Mucosal

Following a single oro-mucosal administration of nabiximols (Sativex®) (four sprays totaling 10.8 mg Δ⁹-THC and 10 mg CBD), mean peak plasma concentrations of both THC and CBD typically occur within 2-4 h. When administered oro-mucosally, blood levels of Δ⁹-THC and other cannabinoids are lower than those achieved by inhalation of the same dose of smoked cannabis, but Δ⁹-THC blood levels were comparable to those seen with oral administration of dronabinol (108,290). Oro-mucosal administration of nabiximols is amenable to self-titration (19, 20, 21, 22).

Rectal

While Δ⁹-THC itself is not absorbed through the rectal route, the pro-drug Δ⁹-THC-hemisuccinate is absorbed; this fact, combined with decreased first-pass metabolism through the rectal route, results in a higher bioavailability of Δ⁹-THC by the rectal route (52-61%) than by the oral route (23, 24, 25, 26, 27). Plasma concentrations of Δ⁹-THC are dose and vehicle-dependent, and also vary according to the chemical structure of the THC ester (26). The rectal route is less desirable but offers an alternative for patients lacking conscious coordination.

Topical

Cannabinoids are highly hydrophobic, making transport across the aqueous layer of the skin the rate-limiting step in the diffusion process (1). No clinical studies exist regarding the percutaneous absorption of cannabis—containing ointments, creams, or lotions. However, some research has been carried out on transdermal delivery of synthetic and natural cannabinoids using a dermal patch (28, 29). Permeability of various cannabinoids vary such that cannabidiol (CBD) and cannabinol (CBN) was found to be 10-fold more permeable through the skin than for Δ⁸-THC (30).

Liposomal Delivery of Cold Medicinal Cannabis Extract by Oral Inhalation

In the 1970s, scientists proposed using liposomes for the delivery of drugs. Today we have a variety of drugs in use given in liposomal form for direct injection into our blood stream.

Liposomes, spherical vesicles, having at least one lipid layer, are formed when surfactant, co-surfactant, emulsifiers and/or minute detergents are added with a fat soluble substance in water. Liposomes can be prepared by disrupting biological membranes such as by sonication. Such a structure formed can be used as a vehicle for the administration of fat soluble pharmaceutical drugs for direct delivery into the blood stream by injection.

Liposomes vary in size, typically in the low microns. We have formulated a medicinal cannabis extract solution with electrolytes suitable for nebulization using the most advanced hand held VMN today that could deliver particle sizes below 5 micron for deposition deep into the pulmonary tree.

Summary Benefit of the Present Invention:

To deliver cannabis oil extract effectively through the most advanced hand held nebulizer on the market today, the extract containing but not limited to entities listed in Appendix A must be emulsified using respirable emulsifiers and/or surfactants that could carry the extracts in a stable liposomal form in a water base solution to be inhaled deep into the lung for deposition and immediate absorption.

VMN technology is not new. Its deployment has steadily improved over the course of the past ten years. But it is only recently that the designs have improved so significantly as evidenced by the filing of Patent Application with the United States Patent and Trademark Office #29/552,887; #29/552,888 and 29/552,885 that such devices could have a broad consumer appeal.

By using the present invention, the cannabis extract is able to be formulated into a deliverable form for use in VMN that can produce particle sizes of less than 5 micron capable of being deeply inhaled to produce the desired effects with a much lower dose. In this format, titration of dose requirement could easily be achieved.

There are three types of aerosol delivering devices:

1: Propellant dependent devices such as meter dose inhalers using hydroxyfluroalkane as the propellant

2: Propellant-free devices such as dry powder inhalers

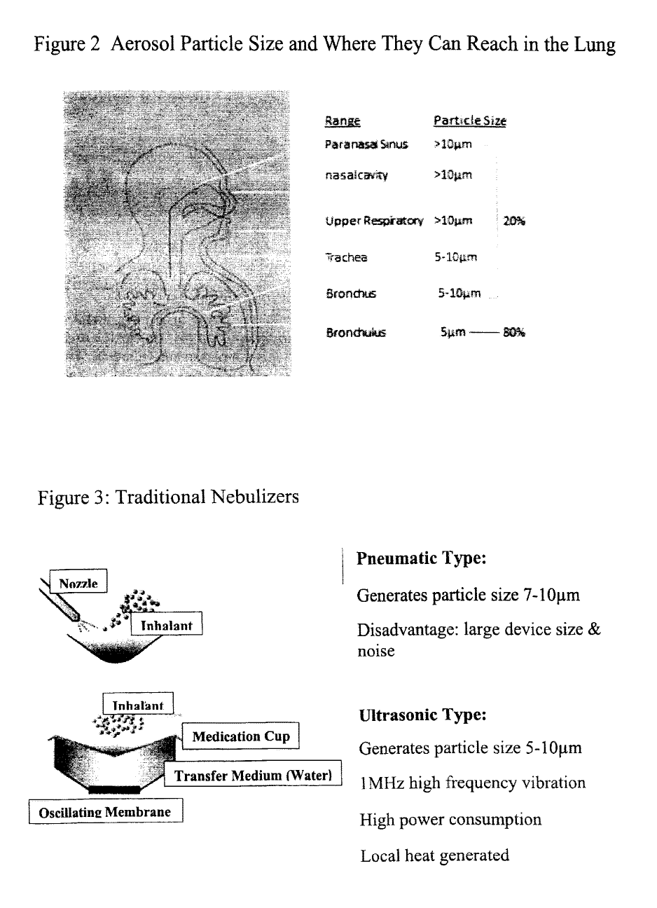

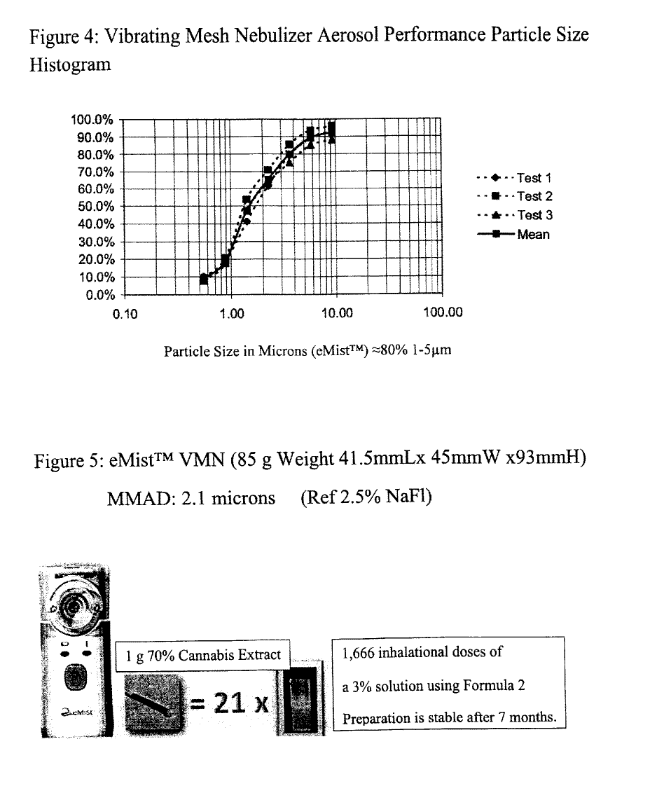

3: Nebulizers: pneumatic type (FIG. 2), ultrasonic type (FIG. 2) (1.6-1.8 MHz, 2.4-2.5 MHz) or the vibrating mesh ultrasonic type (>20 kHz)

While our pharmaceutical formulation of claim 1 could be used in all types of nebulizers, it is with particular interest that we want to formulate a solution that will be efficiently discharged from the mesh ultrasonic type of nebulizers known to nebulize solution particle sizes in the below 5 micron range without clogging up the mesh apparatus. Pulmondary deposition for the traditional nebulizers are inferior to the VMN today (FIG. 3). The VMN uses mesh deformation to push the liquid drug through the mesh. An annular piezo element, which is in contact with the mesh, is used to produce vibration around the mesh, and the liquid drug is in direct contact with the mesh. Holes in the mesh have a conical structure, with the largest cross-section at the cone in contact with the liquid drug. The mesh deforms into the liquid side, thus pumping and loading the holes with liquid. This deformation on the other side of the liquid-drug reservoir ejects droplets through the holes, which can be inhaled by the patient.

Our formulation forms composite structures of liposomes with cannabis extracts emulsified in a stable form using surfactants, electrolytes, emulsifiers and/or co-surfactants and is able to be delivered in particle sizes in the low micrometers for inhalation using VMN. A histogram for the device used in testing our invention is shown in FIG. 3.

While a preferred embodiment of the invention has been described and illustrated above, it should be understood that it is exemplary of the invention is not to be considered as limiting. Additions, omissions, substitutions, and other modifications can be made without departing from the spirit or scope of the present invention. Accordingly, the invention is not to be considered as limited by the foregoing. Various changes and modifications may be made in the concentration, composition and compounding of the formulations described above which fall within the spirit of this invention and all such changes and modifications coming within the scope of the appended claims are embraced thereby.

### Appendix A

### Cannabis May Contain: Cannabinoids, Terpenes and Flavonoids

Sample list of some commonly found cannabinoids, terpenes and flavonoids in cannabis (31):

Phytocannabinoids

THC (Δ-9-tetrahydrocannabinol)

Boiling point: 157° C./314.6° Fahrenheit

Properties: Euphoriant, Analgesic, Anti Inflammatory, Antioxidant, Antiemetic

CBD (cannabidiol)

Boiling point: 160-180° C./320-356° Fahrenheit

Properties: Anxiolytic, Analgesic, Antipsychotic, Anti Inflammatory, Antioxidant,

Antispasmodic

CBN (Cannabinol)

Boiling point: 185° C./365° Fahrenheit

Properties: Oxidation, breakdown, product, Sedative, Antibiotic

CBC (cannabichromene)

Boiling point: 220°/428° Fahrenheit

Properties: Anti Inflammatory, Antibiotic, Antifungal

Δ-8-THC (Δ-8-tetrahydrocannabinol)

Boiling point: 175-178° C./347-352.4° Fahrenheit

Properties: Resembles Δ-9-THC, Less psychoactive, More stable Antiemetic

THCV (Tetrahydrocannabivarin)

Boiling point: <220° C./<428° Fahrenheit

Properties: Analgesic, Euphoriant

Terpenoid Essential Oil Components of Cannabis

β-Myrcene

Boiling point: 166-168° C./330.8-334.4° Fahrenheit

Properties: Analgesic. Anti-Inflammatory, Antibiotic, Anti-Mutagenic

β-Caryophyllene

Boiling point: 119° C./246.2° Fahrenheit

Properties: Anti Inflammatory, Cytoprotective (gastric mucosa), Antimalarial

d-Limonene

Boiling point: 177° C./350.6° Fahrenheit

Properties: Cannabinoid agonist?, Immune potentiator, Antidepressant, Anti-Mutagenic

Linalool

Boiling point: 198° C./388.4° Fahrenheit

Properties: Sedative, Antidepressant, Anxiolytic, Immune potentiator

Pulegone

Boiling point: 224° C./435.2° Fahrenheit

Properties: Memory booster, AChE inhibitor, Sedative, Antipyretic

1,8-Cineole (Eucalyptol)

Boiling point: 176° C./348.8° Fahrenheit

Properties: AChE inhibitor, Increases cerebral blood flow, Stimulant, Antibiotic,

Antiviral, Anti-Inflammatory, Anti-Nociceptive

α-Pinene

Boiling point: 156° C./312.8° Fahrenheit

Properties: Anti-Inflammatory, Bronchodilator, Stimulant, Antibiotic, Antineoplastic,

AChE inhibitor

α-Terpineol

Boiling point: 217-218° C./422.6-424.4° Fahrenheit

Properties: Sedative, Antibiotic, AChE inhibitor, Antioxidant, Antimalarial

Terpineol-4-ol

Boiling point: 209° C./408.2° Fahrenheit

Properties: AChE inhibitor. Antibiotic

p-Cymene

Boiling point: 177° C./350.6° Fahrenheit

Properties: Antibiotic, Anticandidal, AChE inhibitor

Borneol

Boiling point: 210° C./410° Fahrenheit

Properties: Antibiotic

Δ-3-Carene

Boiling point: 168*C/334.4° Fahrenheit

Properties: Anti Inflammatory

Flavonoid and Phytosterol Components of Cannabis

Apigenin

Boiling point: 178° C./352.4° Fahrenheit

Properties: Anxiolytic, Anti Inflammatory, Estrogenic

Quercetin

Boiling point: 250° C./482° Fahrenheit

Properties: Antioxidant, Anti-Mutagenic, Antiviral, Antineoplastic

Cannflavin A

Boiling point: 182° C./359.6° Fahrenheit

Properties: COX inhibitor, LO inhibitor

β-Sitosterol

Boiling point: 134° C./273.2° Fahrenheit

Properties: Anti Inflammatory, 5-α-reductase, inhibitor

### REFERENCE LIST

1: Huestis, M. A. (2007). Human cannabinoid pharmacokinetics. Chem. Biodivers. 4: 770-1804.

2: Uurman, L., Ippel, A. E., Moin, E., and van Gerven, J. M. (2009). Biomarkers for the effects of cannabis and THC in healthy volunteers. Br. J. Clin. Pharmacol. 67: 5-21.

3: Agurell, S., Halldin, M., Lindgren, J. E., Ohlsson, A. and others. (1986). Pharmacokinetics and metabolism of delta 1-tetrahydrocannabinol and other cannabinoids with emphasis on man. Pharmacol. Rev. 38: 21-43.

4: Abrams, D. I., Vizoso, H. P., Shade, S. B., Jay, C. and others. (2007). Vaporization as a Smokeless Cannabis Delivery System: A Pilot Study. Clin. Pharmacol. Ther. 82: 572-578.

5: McClure, E. A., Stitzer, M. L., and Vandrey, R. (2012). Characterizing smoking topography of cannabis in heavy users. Psychopharmacology (Berl). 220: 309-318.

6: Grotenhermen, F. (2003). Pharmacokinetics and pharmacodynamics of cannabinoids. Clin. Pharmacokinet. 42: 327-360.

7: Huestis, M. A. (2005). Pharmacokinetics and metabolism of the plant cannabinoids, delta9-tetrahydrocannabinol, cannabidiol and cannabinol. Handb. Exp. Pharmacol. 657-690.

8: Arter, G. T., Weydt, P., Kyashna-Tocha, M., and Abrams, D. I. (2004). Medicinal cannabis: rational guidelines for dosing. Drugs. 7: 464-470.

9: Gieringer, D. H. (2001). Cannabis “Vaporization”. Journal of Cannabis Therapeutics. 1: 153-170.

10: Gieringer, D., St Laurent, J., and Goodrich, S. (2004). Cannabis vaporizer combines efficient delivery of THC with effective suppression of pyrolytic compounds. Journal of Cannabis Therapeutics. 4: 7-27.

11: Hazekamp, A., Ruhaak, R., Zuurman, L., van, Gerven J. and others. (2006). Evaluation of a vaporizing device (Volcano) for the pulmonary administration of tetrahydrocannabinol. J. Pharm. Sci. 95: 1308-1317.

12: Pomahacova, B., Van der Kooy, F., and Verpoorte, R. (2009). Cannabis smoke condensate III: the cannabinoid content of vaporised Cannabis sativa. Inhal. Toxicol. 21: 1108-1112.

13: Killestein, J., Hoogervorst, E. L., Reif, M., Kalkers, N. F. and others. (2002). Safety, tolerability, and efficacy of orally administered cannabinoids in MS. Neurology. 58: 1404-1407.

14: Walsh, D., Nelson, K. A., and Mahmoud, F. A. (2003). Established and potential therapeutic applications of cannabinoids in oncology. Support. Care Cancer. 11: 137-143.

15: Ne, E. J., Johnson, R. E., Paul, B. D., Mell, L. D. and others. (1988). Marijuana-laced brownies: behavioral effects, physiologic effects, and urinalysis in humans following ingestion. J. Anal. Toxicol. 12: 169-175.

16: Versen, L. L. The pharmacology of THC, the psychoactive ingredient in cannabis. The science of marijuana. New York, N.Y.; Oxford University Press, 2000.

17: GW Pharmaceuticals. Sativex Product Monograph. 2010.

18: Karschner, E. L., Darwin, W. D., Goodwin, R. S., Wright, S. and others. (2011). Plasma cannabinoid pharmacokinetics following controlled oral delta9-tetrahydrocannabinol and oromucosal cannabis extract administration. Clin. Chem. 57: 66-75.

19: Karschner, E. L., Darwin, W. D., McMahon, R. P., Liu, F. and others. (2011). Subjective and physiological effects after controlled Sativex and oral THC administration. Clin. Pharmacol. Ther. 89: 400-407.

20: Ke, D. R., Robson, P., Ho, M., Jubb, R. W. and others. (2006). Preliminary assessment of the efficacy, tolerability and safety of a cannabis-based medicine (Sativex) in the treatment of pain caused by rheumatoid arthritis. Rheumatology. (Oxford). 45: 50-52.

21: Wade, D. T., Makela, P., Robson, P., House, H. and others. (2004). Do cannabis-based medicinal extracts have general or specific effects on symptoms in multiple sclerosis? A double-blind, randomized, placebo—controlled study on 160 patients. Mult. Scler. 10: 434-441.

22: Nurmikko, T. J., Serpell, M. G., Hoggart, B., Toomey, P. J. and others. 2007). Sativex successfully treats neuropathic pain characterised by allodynia: a randomised, double-blind, placebo-controlled clinical trial. Pain. 133: 210-220.

23: Enneisen, R., Egli, A., Elsohly, M. A., Henn, V. and others. (1996). The effect of orally and rectally administered delta 9-tetrahydrocannabinol on spasticity: a pilot study with 2 patients. Int. J. Clin. Pharmacol. Ther. 34: 446-452.

24: Mattes, R. D., Shaw, L. M., Edling-Owens, J., Engelman, K. and others. (1993). Bypassing the first-pass effect for the therapeutic use of cannabinoids. Pharmacol. Biochem. Behav. 44: 745-747.

25: Perlin, E., Smith, C. G., Nichols, A. I., Almirez, R. and others. (1985). Disposition and bioavailability of various formulations of tetrahydrocannabinol in the rhesus monkey. J. Pharm. Sci. 74: 171-174.

26: Sohly, M. A., Little, T. L., Jr., Hikal, A., Harland, E. and others. (1991). Rectal bioavailability of delta-9-tetrahydrocannabinol from various esters. Pharmacol. Biochem. Behav. 40: 497-502.

27: Sohly, M. A., Stanford, D. F., Harland, E. C., Hikal, A. H. and others. (1991). Rectal bioavailability of delta-9-tetrahydrocannabinol from the hemisuccinate ester in monkeys. J. Pharm. Sci. 80: 942-945.

28: Valiveti, S., Hammell, D. C., Earles, D. C., and Stinchcomb, A. L. (2004). Transdermal delivery of the synthetic cannabinoid WIN 55,212-2: in vitro/in vivo correlation. Pharm. Res. 21: 1137-1145.

29: Valiveti, S., Kiptoo, P. K., Hammell, D. C., and Stinchcomb, A. L. (2004). Transdermal permeation of WIN 55,212-2 and CP 55,940 in human skin in vitro. Int. J. Pharm. 278: 173-180.

30: Stinchcomb, A. L., Valiveti, S., Hammell, D. C., and Ramsey, D. R. (2004). Human skin permeation of Delta8-tetrahydrocannabinol, cannabidiol and cannabinol. J. Pharm. Pharmacol. 56: 291-297.

31: Atakan, Z. (December 2012). Ther Adv Psychopharmacol 2(6): 241-254

32: Sen C K, Khanna S, Roy S (2006) “Tocotrienols: Vitamin E Beyond Tocopherols”. Life Sciences 78 (18): 2088-98

33: Das S, Lekl L, Das M et al (2008), “Cardioprotection with Palm Oil Tocofrienols; Comparison of Different Isomers” American Journal of Physiology, Heart and Circulatory Physiology 294 (2): H970-8

34: Khama S, Roy S et al (2005)” Neuroprotective Properties of the Natural Vitamin E alpha-Tocotrienol” Stroke 36 (102258-64)

1. Pharmaceutical formulations of a base solution, suitable for the delivery of various oil based substances, including various concentration and ratio of CBD to THC in a cannabis extract, using a VMN. The formulations comprise of a water base liquid, wherein the liquid is made up of a mixture of added cannabis oil extract having a predetermined amount of a medicinal cannabis compound; and an aerosol precursor with an emulsifier and/or surfactant with electrolytes forming liposomes by sonication.

2. The pharmaceutical formulation of claim 1, wherein the water base liquid can be delivered in an aerosol.

3. The pharmaceutical formulation of claim 1, wherein the water base liquid can be delivered in a liposomal micro-emulsified form.

4. The pharmaceutical formulation of claim 1, wherein the water base liquid is able to be nebulized effectively by a nebulizer including but not limited to a VMN that is capable of delivering respirable solution particles in a Mass Median Aerodynamic Diameter (MMAD) of 2-3 microns, thus enhancing the bioavailability of the medicinal compound for absorption.

5. The pharmaceutical formulation of claim 1, wherein the medicinal cannabis compound of known amount comprises none or one to more cannabinoid(s).

6. The pharmaceutical formulation of claim 1, wherein the medicinal cannabis compound of known amount comprises none or one to more terpene(s).

7. The pharmaceutical formulation of claim 1, wherein the medicinal cannabis compound of known amount comprises none or one to more flavonoid(s) and/or phytosterol(s).

8. The pharmaceutical formulation of claim 1, wherein the known amount of the medicinal cannabis compound is delivered in a single unit dose or in a titratable dose format.

9. The pharmaceutical formulation of claim 1, wherein the aerosol precursor comprises of various entities or a combination thereof of inhalable solvents, water base solutions, emulsifiers and/or surfactants along with cannabis in various concentration with or without added fat soluble supplements.

10. The pharmaceutical formulation of claim 1, wherein the water base liquid formulation nebulizes without any heat source.

11. The pharmaceutical formulation of claim 1, wherein the water base liquid formulation nebulizes without smoke or exhale.

12. The pharmaceutical formulation of claims 1 and 4, wherein the rate of nebulization could be pre-programed and the dose varied by the number of actuation of the VMN, the rate of nebulization and/or the length of time of nebulization per actuation of the device.

13. The pharmaceutical formulation of claim 1, wherein any one or more cannabinoid(s) can be administered separately at separate times, simultaneously or sequentially to another one or more cannabinoid(s).

14. The pharmaceutical formulation of claims 1 and 5, wherein the cannabis extracts could be obtained by, but not limited to, supercritical CO2 extraction, decarboxylation, hydro pressure extraction, volatilization with a heated gas, synthetic and/or biosynthetic reengineering.

15. The pharmaceutical formulation of claim 1 could be mixed using sonication, ultrasonic or micro fluidizer.

16. The pharmaceutical formulation of claim 1 utilizes the addition of sodium salt as in sodium lauryl sulfate or sodium chloride to enhance the nebulization efficiency of the VMN for use with oil base pharmaceuticals such as cannabis extract or hemp oil or combination thereof.

17. The pharmaceutical formulation of claim 1 can vary the percentage and type of lecithin with cannabis extracts in a preferred 1:1 ratio.

18. The pharmaceutical formulation of claim 1 can vary the concentration or purity of the cannabis extract from 0 to 100% and proportionately lowering the alcohol content with increasing purity from 10 to 0%.

19. The pharmaceutical formulation of claim 1 could use hemp oil to dilute concentrated cannabis extract and reduce the alcohol content from 10 to 0%.

20. The pharmaceutical formulation of claim 1 could add 0.001 to 0.003% of benzalkonium chloride as a preservative if it is without alcohol and not in a unit dose format.
